(19) United States
(12) Patent Application Publication
(10) Pub. No.: US 2020/0246089 A1
(43) Pub. Date: Aug. 6, 2020

(54) Device for Guiding a Medical Flexible Shaft

(71) Applicant: Deutsches Zentrum für Luft- und Raumfahrt e.V., Koeln (DE)

(72) Inventors: Christopher Schlenk, Munchen (DE); Julian Klodmann, Munchen (DE); Manuel Dosch, Holm (DE); Florian Alexander Fröhlich, Germering (DE)

(21) Appl. No.: 16/776,945

(22) Filed: Jan. 30, 2020

(30) Foreign Application Priority Data
Jan. 31, 2019 (DE) 10 2019 201 277.9

(51) Int. Cl. A61B 34/30; A61B 34/20; A61B 1/00; A61B 1/01

(52) U.S. Cl. CPC A61B 34/30; A61B 34/20; A61B 2034/301; A61B 1/0016; A61B 1/01; A61B 1/00149

(57) ABSTRACT

The invention relates to a device for guiding a medical flexible shaft, in particular an endoscope shaft, to a body into which the shaft is to be inserted. The device includes a length-variable bridging device and a shaft receptacle connected to the bridging device for fixing the shaft to the bridging device. The bridging device prevents a vibration of the shaft at least at the bodily insertion orifice. The invention also relates to a medical endoscopy system comprising such a device. In addition, the invention relates to a medical endoscopy robot system including an endoscopy system of this kind. Lastly, the invention relates to a method for guiding a medical flexible shaft, in particular an endoscope shaft, to a receiving body. In accordance with the method the shaft tip is moved forwards and/or backwards to the receiving body.

### CROSS-REFERENCE TO RELATED APPLICATION

This application claims priority to German Patent Application No. 10 2019 201 277.9 filed Jan. 31, 2019, the disclosure of which is hereby incorporated by reference in its entirety.

### BACKGROUND OF THE INVENTION

### Field of the Invention

The present disclosure relates to a device for guiding a medical flexible shaft to a body into which the shaft is to be inserted. The present disclosure also relates to an endoscopy system, an endoscopy robot system, and a method for guiding a medical flexible shaft to a body into which the shaft is to be inserted.

### Description of Related Art

In medical applications flexible shafts are used for a wide range of purposes. Medical flexible shafts are constituted in particular by tubes, such as respiratory tubes, suction tubes or catheters, by cables, and particularly preferably by endoscope shafts.

These shafts may be inserted into small bodily orifices. In addition, these orifices may not be easily accessible.

A significant application of medical flexible shafts is constituted by flexible endoscopes, that is to say endoscopes with a flexible shaft. Flexible endoscopes are used for a large number of medical tasks, such as the examination of hollow organs, such as the intestine, bladder, kidneys, etc.

Numerous problems are encountered in the medical application of flexible endoscopes. In particular, these problems are experienced in relation to manual use of the endoscope. For example, in the case of longer examinations in particular, the handling of the endoscope is physically tiring for the operator due to the inherent weight of the endoscope and also the rigidity of the flexible elements.

Furthermore, the operator needs to possess great skill with regard to the coordinated movement of the endoscope tip, since this has to be produced by combined movements of the gripper unit as a whole and the adjustment possibilities, for example levers or setting wheels, on this gripper unit. Due to the complex mapping of the movements, it is difficult particularly for less experienced users to guarantee a full examination of the target area. Clumsy handling of the endoscope may result in the endoscope, for example the technical system, being damaged.

In addition, the operator of the endoscope must remain permanently in the immediate vicinity of the examined object or the patient. This is problematic for example if, during medical interventions, repeated intraoperative X-raying is necessary, for example in the case of endoscopic removal of kidney stones. In such a case the doctor performing the endoscopy may be exposed to the X-rays, which, when considered over the year, may add up to a high amount of X-ray exposure.

Numerous concepts for use of medical flexible endoscopes are known in the literature and will be presented briefly hereinafter and then discussed in detail in respect of certain disadvantages.

ZHANG et al., Robotic assistance for manipulating a flexible endoscope, in Robotics and Automation (ICRA), 2014 IEEE International Conference on, IEE, 2014. p. 5380-5385 describes a robotically guided endoscope for urology. Here, a commercial endoscope is connected via a suitable sterile interface to a robot arm such that this may move the endoscope in three degrees of freedom: advance, rotation about the longitudinal axis, and lever movement in order to angle the tip. The drive for the lever is connected via a snap-action mechanism to the endoscope lever. The interface also has an annular sleeve for supporting the flexible endoscope shaft between the robot and patient body in order to prevent sagging.

RUITER et al., Design and evaluation of robotic steering of a flexible endoscope, in Biomedical Robotics and Biomechatronics (BioRob), 2012 4th IEEE RAS & EMBS International Conferences on. IEEE, 2012. p. 761-767 describes a robotic drive module for a colonoscope (endoscope for colonoscopy) which may be adapted for various hand-held colonoscopes. The robotic drive module consists of a generic drive unit with two servomotors, which is connected via a Bowden cable to the endoscope-specific endoscope holder, which produces the connection to gearwheels in order to angle the endoscope tip. The doctor controls the endoscope functions, for example angling the endoscope tip in two directions, gas insufflation, rinsing, and suctioning, via a remote control which is held directly in the hand in the case of stationary use of the endoscope and may be secured to the endoscope holder in the case of hand-held use of the endoscope. The angling is controlled here by way of a joystick or a touchpad; all other functions are controlled by way of push-buttons.

FANG et al., A motorized hand-held flexible rhino endoscope in ENT diagnoses and its clinical experiences, in Biomedical Robotics and Biomechatronics (BioRob), 2012 4th IEEE RAS & EMBS International Conference on. IEEE, 2012. 5. 853-858 describes a drive system for a hand-held, flexible rhinoendoscope for diagnostic applications in the ear, nose and throat. The overall system consists of a conventional hand-held, flexible endoscope, a manipulator unit for actuating this endoscope in two degrees of freedom, specifically rotation of the endoscope as a whole about the endoscope longitudinal axis and movement of the lever for angling the endoscope tip, as well as a laptop for controlling the manipulator. The two servomotors are controlled via a microcontroller board by PWM.

EP 1859724 A1 describes a mobile holding device for a flexible endoscope with electrically actuated degrees of freedom, to which the endoscope may be secured both during endoscopic interventions and for mounting. This support device has three degrees of freedom for positioning the endoscope drive unit in relation to the basis of the support device, a stabilising device with ball joints for orienting the endoscope, and an endoscope holder, which enables a rotation about the endoscope longitudinal axis. All degrees of freedom are passive and are arrested manually during the endoscopic intervention.

US 2002/0103418 A1 describes an endoscope system consisting of a holding system and an endoscope secured thereto with electrically actuated degrees of freedom. The endoscope may be either a commercial electrically actuated endoscope or a manually actuated endoscope which is driven via an external motor unit. The controller unit for the electrical actuation of the endoscope may be secured by means of suitable mechanisms to the power cable or to the endoscope shaft. The joints of the holding system may be arrested passively and manually. The endoscope is freely rotatable about the endoscope longitudinal axis relative to the holding system.

CN203468740U describes a manipulation system for actuating a flexible endoscope, consisting of a manipulator, a system controller in the user console, a control unit for the manipulator, and a user console. The doctor remotely controls the manipulator with the endoscope clamped therein from the user console by means of joysticks and a touchscreen. The flexible endoscope may be moved in five degrees of freedom (three in translation, two in rotation). The conventional endoscope is clamped in the system via clamping jaws and screws, which on the one hand fix the endoscope as a whole and on the other hand fix the lever for angling the endoscope.

US 2016/0184032 A1 describes a method for configuring a plurality of surgical robot arms in which the arms form a virtual rail. For example, the robotic insertion of a flexible endoscope into the patient may be simplified as a result. By exerting forces onto one or more robot arms, the position and/or orientation thereof may be changed.

US 2012/0065470 A1 describes a robotic system for endoscopes with a tip that may be angled, consisting of a support arm, an endoscope holder, and a displacement unit which may move the support arm. The system as a whole may either be positioned on a suitable stand or may be secured to the side rails of the operating table. The surgeon may control the translation of the endoscope in the axial direction, the rotation of the endoscope about its longitudinal axis, and the angling of the flexible region at the endoscope tip via a control unit. Endoscopic interventions, such as the removal of tumours from the vocal cords, may thus be performed by an individual doctor instead of by two doctors, as was previously the case.

US 2018/0098687 A1 describes a control unit for a flexible endoscope with two degrees for freedom for angling. The user interface consists of a first part for controlling the endoscope shaft, which is connected via a pivot joint to the rest of the housing and is moved by the user using the palm of the hand. The position of this first part is detected, and the corresponding position of the rotary knobs for angling the endoscope shaft is calculated. A finger interface secured movably to the first part of the user interface forms the second part of the user interface in order to control instruments in the tool channel of the endoscope. This finger interface consists of two pads for controlling a gripper degree of freedom, which pads in addition may be rotated relative to the first part and may be moved in translation. The doctor may hereby actuate all three degrees of freedom of a tool for the tool channel. The rotary knobs are moved via two electric motors with subsequent worm gears. The degrees of freedom of the instrument are driven by a second drive unit which allows a translation of the instrument as a whole, a rotation of the instrument as a whole, and an activation of the gripper. The exact design of this drive unit is dependent on the user interfaces of the instrument.

Disadvantages of such concepts will be presented hereinafter:

Hand-held endoscopes with merely two actuated degrees of freedom, such as those according to RUITER et al., FANG et al. and US 2018/0098687 A1, are technically less complex, but also have some disadvantages. It is tiring for the surgeon to hold the actuated endoscope, the endoscope weight increases further as a result of the actuation, the surgeon has to remain permanently by the patient's side, and the endoscope movements are not reproducible subsequently, since the translation of the endoscope is implemented manually (thus, in the case of iterative procedures such as the removal of kidney stones, it is not possible for a known position to be adopted in automated fashion).

Passive support systems for endoscopes, such as those in EP 1859724 A1 and US 2002/0103418 A1, free the surgeon from having to support the weight of the endoscope during the intervention. With use of an electrically actuated endoscope or a manually actuated endoscope which is actuated via an external drive unit, the surgeon may control the angling of the endoscope tip and also the endoscope functions such as gas insufflation, rinsing and suctioning via a hand controller. The insertion and the advance of the endoscope shaft inside the patient, however, are still implemented manually, as is the rotation of the endoscope about its longitudinal axis. The endoscope movement is therefore not reproducible, and the surgeon must remain permanently by the patient's side.

The existing actuation systems which control all three relevant degrees of freedom (angling of the endoscope tip, rotation about the longitudinal axis of the endoscope, translation of the endoscope), for example as in ZHANG et al., CN 203468740 U and US 2012/0065470 A1, are specialised systems for flexible endoscopy which are extremely complex and costly. In addition, they do not offer any possibility for precise guidance of the endoscope tip, which would be absolutely necessary with automatic insertion of the endoscope into the patient. The annular sleeve described in ZHANG et al. damps vibrations of the flexible endoscope shaft centrally, however there are still undesirable vibrations of the tip of the shaft. At the same time, the maxim insertion length into the patient and thus the working area of the endoscope is hereby limited.

In the method described in US 2016/0184032 A1 the use of robot arms to support the flexible endoscope shaft represents a very high outlay, for example cost for robot arms, and additional time outlay for the pre-operative preparation of the robotic system, etc. In addition, the robot arms block space in the vicinity of the patient and thus in some circumstances impede the access of the medical personnel to the patient.

### SUMMARY OF THE INVENTION

The object of the invention is to create a device for guiding a medical flexible shaft to a body into which the shaft is to be inserted, an endoscopy system, and an endoscopy robot system, wherein the guidance of the flexible shaft is improved. A further object of the invention is to create a method for guiding a medical flexible shaft to a body into which it is to be inserted, in which method the guidance of the flexible shaft is improved.

The object is achieved in accordance with the invention by a device having the features of claim 1, an endoscopy system having the features of claim 14, an endoscopy robot system having the features of claim 17, and a method having the features of claim 18.

The device according to the invention is a device for guiding a medical flexible shaft to a body (referred to hereinafter as the “device”). The body is a body into which the shaft is to be inserted, preferably a patient. The device is preferably a device for guiding at least the tip of the shaft, particularly preferably for guiding the entire shaft. Here, the guidance of the shaft means preferably on the one hand a bridging of the shaft, that is to say in particular a directed holding of the shaft. This guidance occurs in particular starting from a base of the shaft, that is to say a starting point of the shaft, for example an endoscope gripper unit. The guidance is preferably performed as far as a body into which the shaft is to be inserted. Alternatively or additionally the guidance of the shaft means in particular a movement of the shaft towards and/or away from a body into which the shaft is to be inserted. The guidance is preferably performed along a defined, in particular single, movement path, in particular in the form of a straight line or a uniform curve. The shaft is a medical shaft, in particular a tube, such as a respiratory tube, a suction tube or a catheter or the like. It is particularly preferable that the shaft is an endoscope shaft of a medical flexible endoscope. The device has a length-variable bridging device. It is preferable that the bridging device is length-variable in translation, particularly preferably linearly. It is also possible that the bridging device is length-variable along a preferably arched curve. In particular the bridging device is extendable and retractable and/or may be folded out and collapsible. The device also have a shaft receptacle connected to the bridging device. The shaft receptacle is preferably embodied in such a way that it allows the shaft to be fixed to the bridging device. Here, fixing means in particular a holding or supporting of the shaft. The fixing is preferably embodied here in such a way that it on the one hand permits a rotation of the shaft and/or on the other hand permits a proximal and/or distal movement of the shaft. Here, distally describes a movement towards the body into which the shaft is to be inserted, wherein proximally describes the opposite direction accordingly. In particular the fixing is embodied in such a way that the shaft is fixed, that is to say is firmly held in place, at right angles to the proximal-distal direction. In other words the shaft is preferably fixed by means of the shaft receptacle in such a way that no radial movements of the shaft are possible in accordance with the proximal-distal direction. The shaft receptacle preferably has at least one hook and/or at least one eyelet and/or at least one bore. If the shaft receptacle for example has at least one eyelet, through which the shaft is guided, the shaft may thus still be rotated and moved proximally as well as distally, that is to say displaced back and forth. The eyelet, however, prevents the shaft from being able to be moved radially, that is to say in other words for example vertically or horizontally. The length-variable bridging device prevents a vibration of the shaft, in particular of the tip of the shaft, at least at the point of insertion into the body. The point of insertion into the body is in particular the orifice into which the shaft is to be inserted into the body. In this case, preventing the vibration in particular does not mean a complete elimination of vibration, but instead a minimisation of vibration or damping of vibration such that the shaft may be easily accurately inserted into the bodily insertion orifice. An accurate insertion of this kind preferably occurs if the shaft is moved in translation starting from the base of the shaft. For example, it is therefore nevertheless possible that there might be a certain vibration on account of the construction of the bridging device. An alternative definition for preventing vibration is that the length-variable bridging device performs a defined positioning of the shaft, in particular the tip of the shaft, in preferably three-dimensional space, in such a way that the shaft may be inserted in a targeted manner into the point of insertion into the body. An exact positioning and/or exact determination of the position of the endoscope shaft, in particular of the endoscope tip, may advantageously be implemented with the device according to the invention. Endoscope movements are preferably reproducible by means of the device according to the invention.

The length-variable bridging device preferably has a scissor mechanism and/or a telescope mechanism and/or a bellows mechanism. If the device has a scissor mechanism, it is preferred that at least the first two proximal joints are guided or mounted parallel to the proximal-to-distal direction. By means of such a mounting, undesirable rotations of the scissor mechanism are prevented. The scissor mechanism is preferably a two-dimensional scissor mechanism. For example, it is a scissor mechanism as is often used for wall mirrors. The scissor mechanism is particularly preferably a three-dimensional scissor mechanism. In this case it is a scissor mechanism as is often used for lifting platforms.

It is preferred that the shaft receptacle is designed in such a way that the shaft may be received at least at the distal end of the bridging device. To this end, the shaft receptacle is preferably arranged in the region of the distal end of the bridging device. It is particularly preferred that the shelf receptacle is designed in such a way that the shaft may be received over the entire length of the bridging device. Here, the shaft receptacle is preferably formed in such a way that the shaft is guided parallel to the bridging device. Here, “parallel” does not necessarily mean that the bridging device and/or the shaft describe a straight line, but that the bridging device and shaft describe an identical shape oppositely to one another. In this regard it may preferably also be said that the bridging device and shaft represent substantially parallel curves.

In a preferred embodiment the shaft receptacle is formed in such a way that the shaft may be guided at least partially within the bridging device or runs through the bridging device. This may preferably be realised in such a way that the bridging device is hollow, at least in part, such that the shaft may run through the bridging device. If the bridging device for example comprises a telescope mechanism, it is thus preferred that the individual telescope elements, preferably telescope cylinders, are hollow. It is preferably also possible that the telescope elements have a prism shape, in particular cuboid or cube form, such that a rotation of the telescope elements relative to one another is hereby preferably prevented. It is preferred that the bridging device has at least one bore so that the shaft is guidable through this at least one bore. If the bridging device comprises a scissor mechanism, it is thus preferred that at least one central rod of the scissor mechanism has a bore.

In particular, the device comprises a drive device for activating the device. The drive device preferably comprises at least one drive unit, wherein the at least one drive unit is in particular a motor, preferably an electric motor, particularly preferably a servomotor. It is particularly preferred that a preferably separate drive unit is used for each degree of freedom to be activated. In a preferred embodiment the drive device is connected to the device in such a way, for example via a plugged connection, that the drive device is easily separable, in particular by hand, from the device. In particular a complete sterilisation, for example by means of autoclaving of the device, is possible in this way, without the motor being damaged or without the motor requiring a special design, for example by means of a sealed housing.

It is preferred that the drive device activates the bridging device. This activation of the bridging device is in particular an extension and/or retraction of the bridging device.

The drive device preferably activates the shaft and/or an endoscope comprising the shaft, additionally or alternatively to the activation of the bridging device. In the event of an activation of the endoscope it is preferred that the drive device directly or indirectly rotates the endoscope shaft or the entire endoscope inclusive of endoscope shaft. The above-described rotation of the endoscope as a whole, inclusive of endoscope shaft, is particularly preferred, since the endoscope shaft and endoscope gripper units are rigidly connected in the case of most endoscopes. Alternatively or additionally to the rotation, it is preferably possible that the drive device indirectly or directly activates one or more endoscope functions, such as in particular angling functions of the endoscope.

It is preferred that the device comprises a connection device. The connection device is in particular formed in such a way that it connects the device, preferably structurally, to a shaft and/or to a shaft base connected to the shaft. The shaft base is preferably an endoscope gripper unit. Alternatively or additionally, the connection device connects the device preferably to a support, wherein this is preferably a robot.

In a preferred embodiment the connection device comprises a coupling for transmitting an activation to the shaft and/or to an endoscope. The activation to be transmitted is in particular constituted by activations of the drive device. By means of the coupling, the shaft and/or an entire endoscope may preferably be rotated, in particular starting from the drive device, and/or angling functions may be triggered. It is particularly preferred that the coupling is designed to transmit the activation to actuation elements of the endoscope. Here, the coupling is in particular designed in such a way that it couples to actuation elements of the endoscope. The actuation elements to be coupled are in particular rotary wheels and/or levers and/or switches and/or buttons or the like. If, for example, a standard hand-held medical endoscope is guided by means of the device, the coupling may thus couple to actuation elements of the endoscope for example for angling and/or rotating the endoscope shaft, such that in particular the drive device actuates the actuation elements of the endoscope and the various functions are triggered in this way.

It is preferred that the connection device comprises a robot adapter, in particular a robot arm adapter, for connecting the device to a robot. The robot adapter preferably has at least one interface for, in particular, electrical and/or hydraulic energy transmission and/or communication. If the device is therefore connected to a robot or a robot arm, the device on the one hand may thus be guided by this robot or robot arm. Due to the guidance via a robot or robot arm, a precise and/or reproducible movement over large distances in up to six degrees of freedom is possible in particular. On the other hand, it is hereby possible to transmit signals from the robot or robot arm to the device. In particular, the device, preferably the drive device, may be controlled and/or supplied with energy in this way. The device in this way may be mounted in particular at tool interfaces of commercial robots. These should preferably have at least six degrees of freedom and should have suitable safety features, for example low mass and/or integrated torque sensors for detecting external forces, for use in the immediate vicinity of patients and doctors. A robot with such characteristics is for example the KUKA iiwa. Depending on requirements, these robots may be mounted directly on the operating table, on a separate trolley, or on a ceiling mount.

It is preferred that the bridging device extends at least starting from a proximal end of the shaft. Alternatively or additionally, the bridging device extends at least starting from a shaft base. It is furthermore preferred that the bridging device extends at least as far as the bodily insertion orifice into which the shaft is to be inserted, or alternatively or additionally at least as far as the distal end of the shaft. The bridging device preferably extends in translation, particularly preferably linearly. The shaft base is preferably an endoscope gripper unit or a catheter bottle or a catheter bag or the like. The device therefore allows a bridging or a guided connection between the proximal shaft end or shaft base and distal shaft end or bodily insertion orifice for the shaft, preferably over the entire shaft length. It is possible that the bridging device extends beyond the proximal shaft end of the shaft base and/or beyond the distal shaft end or the bodily insertion orifice.

The device preferably comprises a control device for controlling the device remotely. In particular, the control device comprises at least one transmitting and/or receiving device. The control device is in particular a device-side remote control device. The control device is preferably controllable by an external user-side operating device. The control device is in particular designed in such a way that it allows the drive device to be controlled, in particular remotely, for example from an adjoining room in relation to the device. The transmitting and/or receiving device is preferably a wireless and/or wired device. For example in the case of hand-held endoscopes, the endoscope tip is moved by a combination of movement of the endoscope gripper unit in space, in particular to advance and retract the endoscope, rotation of the endoscope gripper unit about the endoscope longitudinal axis (for rotation of the angling plane), and actuation of 1-2 levers/setting wheels on the endoscope gripper unit (in order to angle the endoscope tip in one or two planes). The relationship between these movements and the movements of the endoscope tip is complex and unintuitive, and therefore the doctors performing the intervention require a lot of experience in order to manoeuvre an endoscope of this kind. The movement, in particular translation, preferably for insertion of the endoscope shaft, may be performed preferably in this case by a robot or robot arm. This robot or robot arm is in this case in particular likewise remotely controlled, such that improved application of the endoscope as a whole may preferably result. It is preferred that a robot or robot arm connected to the device may likewise be remotely controlled by means of the control device and/or the operating device. The use of the endoscope may be simplified on account of the remote control by means of the control device. The movement of the endoscope tip in Cartesian space may thus preferably be commanded, in particular following determination of the inverse kinematics of the system. The control commands may be generated by software and/or may be generated by the operator by means of an input apparatus, in particular a tablet/smartphone with integrated position sensor, spacemouse, joystick, gamepad, or gesture recognition system, preferably by means of a Leap Motion Controller, etc.

It is preferred that the device, in particular the bridging device, has autoclavable materials for sterilisation. It is particularly preferred that the device, in particular the bridging device, consists at least externally of materials of this kind. Here, the use of metals and/or plastics is preferred. Autoclaving with hot steam is the most frequently used sterilisation method for reusable devices. Thus, practically all clinics have appropriate facilities for autoclaving. It is particularly preferred that the bridging device consists exclusively of autoclavable materials.

The device, in particular the bridging device, preferably has a holding device for, preferably complete, fixing of the shaft with the device, such that no relative movements between the shaft and device are possible. The holding device is in particular releasable, such that the fixing is temporary. The holding device is preferably provided at the distal end of the bridging device. The holding device preferably comprises a clamp, which in particular is releasable. It is preferred that the device, in particular the bridging device, comprises a device for detecting the pulling and/or pushing force of the moved shaft, in particular in order to avoid a tearing and/or clinking of the shaft. By means of a holding device of this kind, it is possible to move the shaft or the tip of the shaft by means of the bridging device, in particular to move it forwards and/or backwards. For example, the shaft is fixed with the retracted bridging device via the holding device and the bridging device is then extended, such that a joint advance of the bridging device and of the shaft occurs.

In the at least partially extended state, the holding device is then released and the bridging device is subsequently retracted. The shaft remains in its position relative to the bridging device. The shaft therefore does not move back together with the bridging device. This principle in other words corresponds for example to the routine approach in which a cord is inserted into the neck of a sports bag or the like. The tip of the shaft is but retracted in an opposite procedure, accordingly.

The endoscopy system according to the invention comprises an above-described device for guiding a medical flexible shaft to a body into which the shaft is to be inserted. In addition, the endoscopy system comprises an endoscope, in particular a flexible endoscope. The endoscope is preferably an endoscope having one or more features of the shaft and/or of the endoscope that have been described above under the definition of the device for guiding a medical flexible shaft to a body into which the shaft is to be inserted. The endoscopy system is designed in such a way that the bridging device receives the endoscope shaft. The bridging device preferably guides the shaft at least starting from the proximal shaft end, alternatively or additionally at least starting from the shaft base. In particular, the bridging device guides the shaft at least as far as the distal shaft end, alternatively or additionally at least as far as a body into which the shaft is to be inserted. Here, the guidance of the endoscope shaft means in particular a preferably longer-lasting, fixedly held bridging of the endoscope shaft between endoscope gripper unit and receiving body. Alternatively or additionally, the guidance means a movement of the endoscope tip up and/or down in relation to a receiving body. The shaft is preferably guided over the entire shaft length.

The endoscopy system preferably comprises a support for fixing of the endoscopy system in a room. It is particularly preferred that the endoscopy system comprises a single, that is to say an individual support. In other words, the support may be a type of standing leg and/or a carrier, which connects the endoscopy system to a room, for example to the floor of a treatment room. It is preferred that the support is movable within the room, such that the endoscopy system may preferably be moved within the room via the support. In particular, the support is a robot arm, which on the one hand is connected to the room and on the other hand to the endoscopy system. Due to the movability of the robot arm, preferably via robot arm members, the endoscopy system may move within the room, however the endoscopy system is likewise fixed to the room via the robot arm. On the other hand, the support may also be a type of trolley, such that the endoscopy system is displaceable within the room. A combination of robot arm and trolley is also possible. In the case of such a combination of robot arm and trolley, it is preferred that the trolley is not moved into operatively, so as not to impede the reproducibility of the intervention. The trolley may then be used in particular to position the system as a whole in the operating theatre, or to move the system as a whole between operating theatres.

It is preferred that the endoscopy system comprises an operating device, preferably for remote control of the device. The operating device is in particular a user-side remote-controlled device. The operating device preferably comprises an imaging means, for example a monitor and/or imaging spectacles. By way of an imaging means of this kind it is in particular possible to transmit images of the endoscope, in particular an endoscope camera, to an operator. It is preferred that the operating device, additionally or alternatively to the imaging means, comprises at least one input device, such as a joystick and/or a tablet and/or a smartphone and/or a spacemouse and/or a gesture recognition system (for example Leap Motion) and/or a gamepad and/or a keypad and/or a mouse, wherein the at least one input device particularly preferably comprises a position sensor. The operating device therefore in particular comprises the at least one corresponding counter piece to the control device. The control device and operating device preferably together constitute the remote controller of the endoscopy system or of the device. It is preferred that the control device and operating device are matched to one another in respect of hardware and/or software. Here, the expression “in respect of hardware” in particular means the type and/or configuration of the cables or the type of wireless transmission. The expression “in respect of software” means in particular a matching of the drivers for the operating and control device. It is preferred that the remote controller is computer-assisted, and in particular executes predefined commands independently. Predefined commands of this kind are preferably motion patterns and/or independent function executions.

It is preferred that the endoscopy system comprises an entry assistance device at the receiving body. The entry assistance device is preferably a type of funnel, the small opening of which point towards the bodily orifice and/or is inserted into same, and the large opening of which points in the opposite direction, such that the insertion of the tip of the shaft is facilitated. It is preferred that the entry assistance device comprises optical markers in order to enable a tracking of the entry assistance device y a suitable tracking system.

The endoscope robot system according to the invention comprises an endoscope system according to the invention as described above and also a robot arm, in particular a lightweight robot. The device for guiding a medical flexible shaft to a body into which the shaft is to be inserted is connected to the robot arm, preferably at a tool interface of the robot arm. The robot arm or the tool interface preferably have a hollow axis. It is particularly preferred that the endoscope robot system comprises merely one individual robot arm. The endoscope is preferably connected to the tool interface, oriented at a right angle or in parallel, via the device for guiding a medical flexible shaft to a body into which the shaft is to be inserted. The robot is preferably controllable via the remote controller.

The method according to the invention is a method for guiding a medical flexible shaft, in particular an endoscope shaft, to a body into which the shaft is to be inserted. The flexible shaft is guided by means of the method according to the invention in particular merely as far as the physical boundary or just before the physical boundary of the receiving body. In the method, at least the tip of the shaft is guided forwards or backwards to the receiving body preferably in translation, particularly preferably linearly. In particular, the tip of the shaft is moved forwards and/or backwards to the receiving body. The tip of the shaft is preferably advanced and/or retracted. In particular, the guidance forwards and/or backwards and/or the movement forwards and/or backwards of the tip of the shaft is realised by means of a length-variable bridging device. It is preferred that the length-variable bridging device or the use of the length-variable bridging device is realised in accordance with one or more features of the above definition of the device for guiding a medical flexible shaft to a body into which the shaft is to be inserted and/or for guiding the endoscopy system and/or the endoscopy robot system. The movement of the tip of the shaft forwards and/or backwards is preferably a movement of the tip of the shaft forwards and/or backwards via the bridging device, wherein the bridging device is preferably already stretched out lengthwise, in particular completely. The bridging device preferably forms a type of rail guide for the shaft or the tip of the shaft, wherein the shaft is moved along the rail guide by way of a pushing and pulling of the shaft, in particular acting externally of the bridging device, for example by a user. Alternatively, the movement of the tip of the shaft forwards and/or backwards is a movement of the tip of the shaft up and/or down by means of the bridging device, such that the bridging device preferably guides and/or moves the shaft. Here, it is preferred that at least part of the endoscope shaft, in particular the entire endoscope shaft, is received by the bridging device, for example is placed inside the bridging device, already prior to the movement up and/or down. It is possible that, during the movement, at least the endoscope tip is arranged in front of the insertion orifice in a guided manner, in particular via the bridging device. As a result of this arrangement of the endoscope tip, for example by a medical user, the endoscope may be inserted as a next step remotely, wherein vibrations of the endoscope tip are prevented. In the case of a subsequent insertion, which in particular is not part of the method, the bridging device is preferably collapsed. Alternatively to the above-described manual arrangement of the endoscope tip in front of the insertion orifice, it is possible that the endoscope tip is arranged by means of the bridging device, wherein the endoscope tip is preferably moved towards the insertion orifice by the bridging device. A corresponding retraction is also possible.

It is preferred that within the scope of the method the flexible shaft is guided along a line from a shaft base, in particular an endoscope gripper unit, to the receiving body. This line form may be on the one hand linear, but on the other hand also curved, for example in the form of an arc. If the guidance is implemented for example by means of a straight telescopic rod, the shaft is also guided in a straight line linearly. If the guidance is implemented by means of a curved or arched telescopic rod, the shaft is thus likewise guided in arched form. The shaft is particularly preferably guided parallel to the length-variable bridging device.

It is preferred that the method is performed by means of an above-described device for guiding a medical flexible shaft to a body into which the shaft is to be inserted and/or by means of an above-described endoscopy system and/or by means of an above-described endoscopy robot system. It is likewise preferred that the method has one or more method features in accordance with the above description of this device and/or these systems.

Within the scope of the method the tip of the shaft is preferably moved forwards and/or backwards by one or more method steps in accordance with the above-described procedure for “inserting a cord into the neck of a sports bag”.

### BRIEF DESCRIPTION OF THE DRAWINGS

The invention will be described in greater detail hereinafter on the basis of preferred embodiments with reference to the drawings.

In the drawings:

FIG. 1 shows a perspective view of an endoscope of the prior art,

FIG. 2a shows a schematic detailed view of the endoscope tip from FIG. 1 in region II in a starting position,

FIG. 2b shows a schematic view of the endoscope tip from FIG. 2a in an angled position,

FIG. 3 shows a schematic view of the principle of the endoscope guidance by means of a robot,

FIG. 4a shows a schematic side view of an embodiment of an endoscopy system according to the invention with an embodiment of a device according to the invention in one position,

FIG. 4b shows a schematic side view of the embodiment from FIG. 4a in a further position,

FIG. 5 shows a schematic view of a further embodiment of an endoscopy system according to the invention with an embodiment of a device according to the invention,

FIG. 6 shows a schematic side view of a further embodiment of an endoscopy system according to the invention with an embodiment of a device according to the invention,

FIG. 7 shows a schematic side view of an embodiment of an endoscopy robot system according to the invention with an embodiment of an endoscopy system according to the invention with an embodiment of a device according to the invention,

FIG. 8 shows a perspective view of a further embodiment of a device according to the invention,

FIG. 9 shows a sectional view of the device from FIG. 8,

FIG. 10 shows a perspective view of an embodiment of a drive system and of a connection device of a device according to the invention,

FIG. 11 shows a sectional view of the depiction from FIG. 10, and

FIGS. 12a-12c show schematic side views of further embodiments of endoscopy robot systems according to the invention.

### DESCRIPTION OF THE INVENTION

Similar or identical components or elements are identified in the figures by like reference signs or variants of these reference signs. In particular for improved clarity, elements already identified are preferably not provided in all figures with a reference sign.

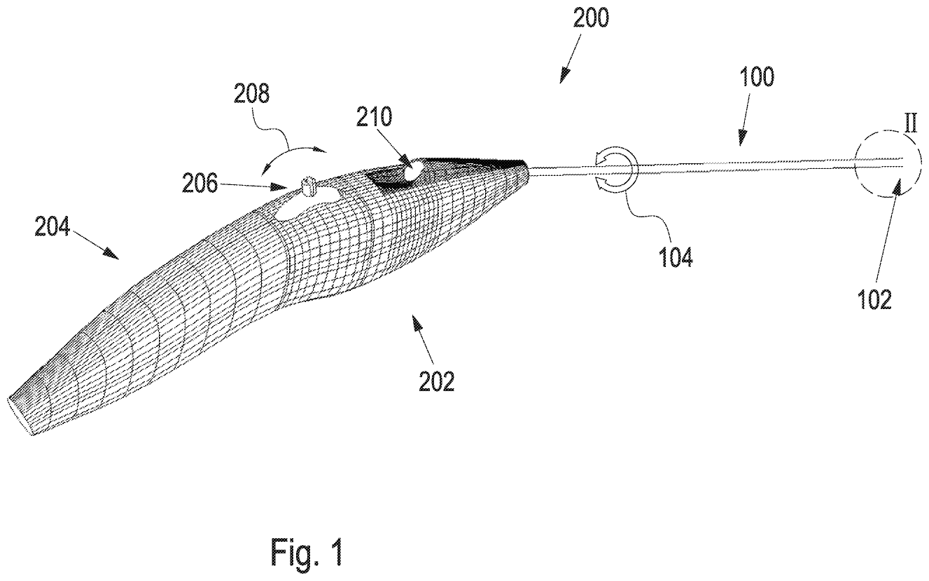

FIG. 1 shows an endoscope 200 with flexible shaft 100 for the hand-held use according to the prior art. The shown endoscope 200 is a flexible ureteroscope, that is to say an endoscope for urological interventions.

For example, with manual use of the endoscope 200, a doctor holds the gripper piece 204 of the endoscope gripper unit 202 in his hand and inserts the shaft 100, at least in part, into the patient for the endoscopic intervention. By movement of the actuation element 206 (along the movement arrow 208), the endoscope tip 102 may be curved in a plane. Various tools, for example optical waveguides for a laser and/or collection cups for removing bladder stones, etc. may be inserted through the working channel 210. The plane in which the endoscope tip curves may be varied preferably by rotating the entire endoscope about its longitudinal axis (see movement arrow 104). The shown endoscope 200 therefore has two degrees of freedom: rotation about the longitudinal axis of the endoscope 200 or of the endoscope shaft 100, and angling of the endoscope tip 102.

Whereas the number of degrees of freedom is usually identical in different hand-held endoscopes for the same medical application, the geometry, in particular of the gripper piece 204, and/or the geometry and the position of the actuation element 206 vary. In particular the actuation element 206 has a lever and/or a rotary head and/or, as shown, a setting wheel.

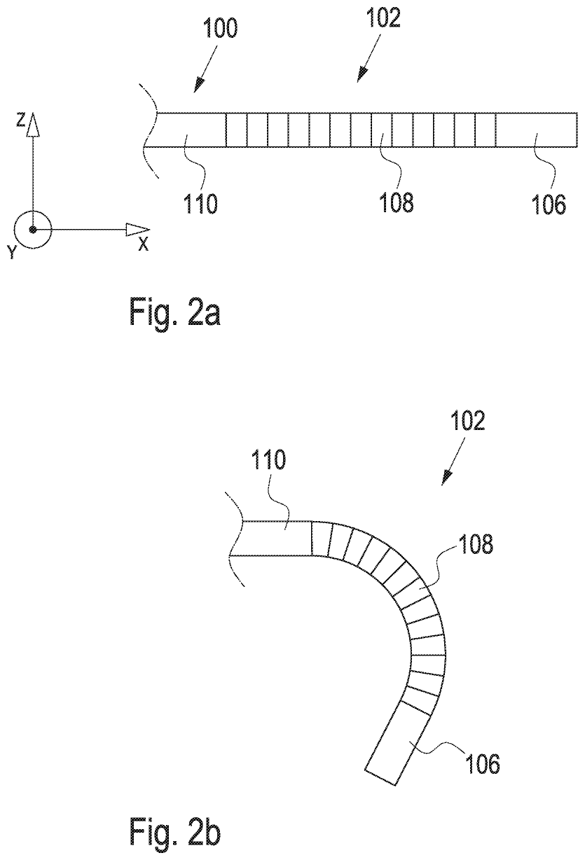

By actuating the actuation element 206, a flexible region of the endoscope tip or in the vicinity of the endoscope tip may be angled (see FIGS. 2a and 2b). The movement is transmitted in particular via wire cables and/or rod-activated mechanisms. If the endoscope 200 is an endoscope with a small shaft diameter, for example for use in the urethra or in the nose and throat area, the shaft has in particular only one angling degree of freedom. If the endoscope 200 has a larger shaft diameter, for example for use in the gastrointestinal tract, the shaft 100 may preferably have two angling degrees of freedom orthogonal to one another. The angling degrees of freedom preferably allow an angling from the zero position in the x- and/or y-direction. The combination of the angling in the x- and y-direction is particularly preferred.

FIG. 2a shows a detailed view of the endoscope tip 102 from FIG. 1.

The region of the endoscope tip 102 comprises a movable angling region 108, which in particular may be curved in a plane via the actuation element 206 (see FIG. 1). This curvature of the angling region 108 is realised in particular by means of cables and/or rods running in the endoscope shaft 100, wherein it is particularly preferred that this is realised by means of Bowden cables. During a curvature of the angling region 108, it is preferred that the rest of the shaft 110 and the endoscope tip head 106 is uncurved, in other words is thus rigid, apart from the general flexibility of the shaft 100.

FIG. 2b shows the endoscope tip 102 from FIG. 2a in the curved or angled state.

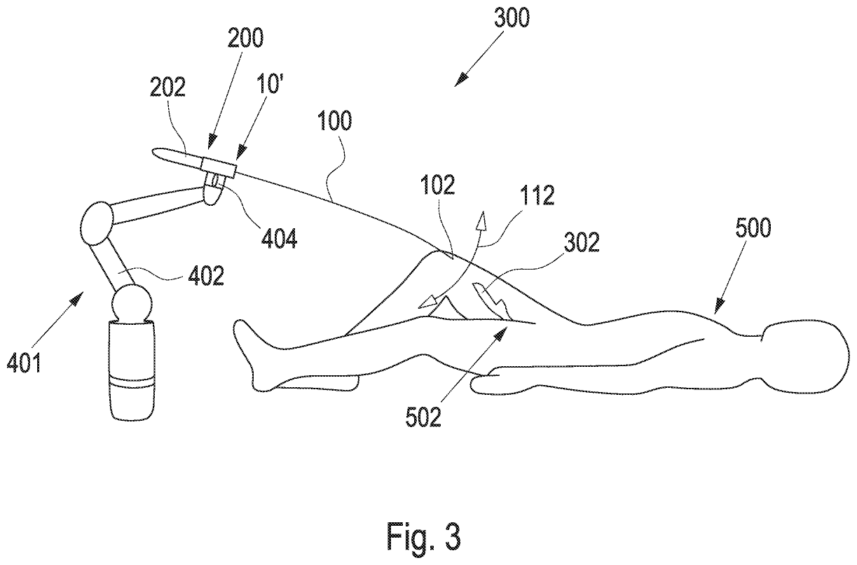

FIG. 3 shows an exemplary principle of an endoscopic intervention with a flexible endoscope 200 guided by a robot arm 402 of a robot 401. In the present case the robot arm 402 corresponds substantially to the robot 401. The endoscope is in particular an endoscope according to the embodiment from FIG. 1.

The endoscope 200 is connected to the robot 401 via a device 10′ to the last element 404 of the robot arm 402. The robot 401 is positioned within the room at a suitable point in the vicinity of the patient 500. Here, the robot 401 may be fastened in particular as required to a suitable support system, for example side rails of an operating table, a movable trolley, a ceiling mount, or the like. The use of such support systems merely for pre-operative positioning, preferably of the robot 401, is preferred so that there is no intra-operative displacement. Optionally, a pre-operative movement is alternatively also conceivable.

In particular the endoscope 200 is releasably connected to the guide device 10′, which is in turn fastened to the last element 404 of the robot, wherein this is in particular a tool interface 404 of the robot. The guide device 10′ is in particular designed in such a way that this may activate on the one hand a rotation of the endoscope shaft or the entire endoscope inclusive of endoscope shaft, and on the other hand, in particular with a coupling to the actuation element of the endoscope 200, activates an angling of the endoscope tip 102.

In particular, for facilitated insertion of the endoscope shaft 100 into the patient body 500, an entry assistance device 302 is arranged at the bodily insertion orifice 502. This assistance device 302, as shown here, corresponds substantially to the design of a funnel. It is preferred that the entry assistance device 302 comprises optical markers (not shown) in order to enable a tracking of the entry assistance device 302 by suitable tracking system (not shown).

Due to the flexibility of the endoscope shelf 100 and/or the length of the endoscope shaft and the resultant distance of the mounting or fixing the endoscope shaft on the endoscope gripper unit 202, vibrations result in the region of the endoscope tip 102 and are shown by way of the movement arrow 112. These vibrations lead to problems during the insertion and/or use of the endoscope.

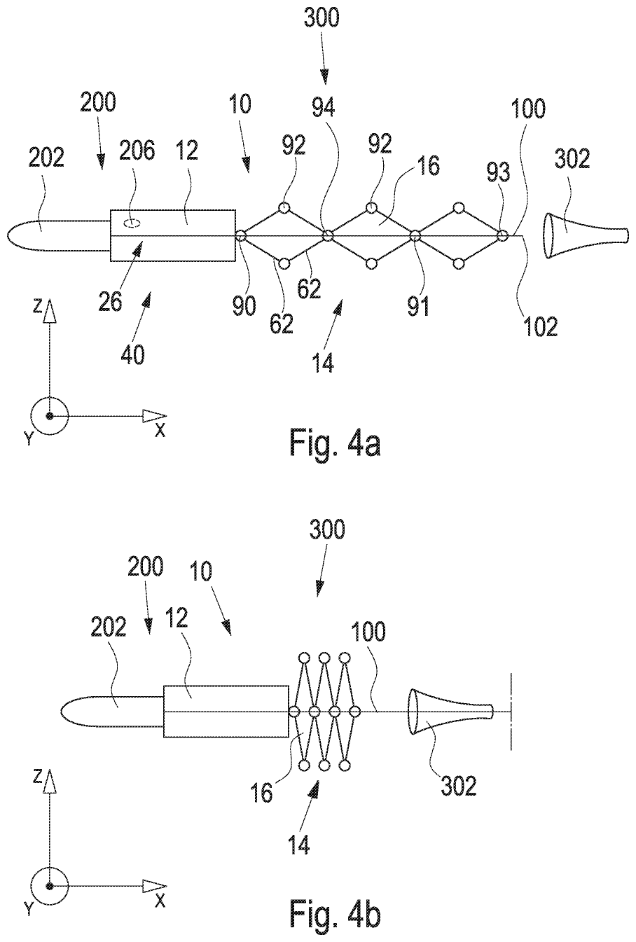

FIG. 4a shows an embodiment of an endoscope system 300 according to the invention with an embodiment of a device 10 according to the invention for guiding a medical flexible shaft to a body into which the shaft is to be inserted.

The device 10, as shown, comprises a connection device 12 for connecting the device 10 to the shaft 100. As is shown, the shaft 100 is an endoscope shaft 100 of the endoscope 200. The connection device 12 is thus connected to the endoscope 200 and hereby to the endoscope shaft 100. The connection device 12, as shown, thus comprises an endoscope carrier 44 connecting the endoscope 200 to the device 10, wherein, as is shown, this is a receptacle for the endoscope 200 in the connection device 12. In addition, the connection device 12 in particular comprises a coupling 26, such that the device 10 hereby couples preferably to the actuation element 206 of the endoscope 200 and may thus activate same. It is likewise possible (not shown) that the connection device 12 comprises a robot adapter 50, via which the device may be connected to a robot arm 402 or a robot 401. As is shown, the connection device 12 preferably comprises the coupling 26, endoscope carrier 40 and in particular (not shown) robot adapter 50 within a component, or performs corresponding functions. In particular, the entire endoscope 200, inclusive of endoscope gripper unit 202 and endoscope shaft 100, rotates about the longitudinal axis by way of the connection device 12.

As is shown, the device 10 comprises a bridging device 14, with, in particular two-dimensional or three-dimensional, scissor mechanism. The scissor mechanism consists of a plurality of scissor bars 62, which are connected movably to one another via joints 90, 91, 92, 93, 94. The scissor mechanism 14 (see FIG. 4b) may be extended and retracted in this way.

It is shown that the first central joint 90 is fixedly connected to the connection device 12 or mounted such that the first central joint 90 preferably allows only rotary movements of the scissor bars 62 connected to the joint 90, in order to enable the folding function of the scissor mechanism 14. It is preferred that the second central joint 94 (not shown) is mounted in such a way that it is mounted parallel to the X-axis, such that in particular no movement of the joint 94 is possible in the Z-direction and/or Y-direction. A linear deflection of the scissor mechanism 14, in particular along the X-direction, is possible in this way. A rotation of the scissor mechanism 14 about the Y-axis is preferably prevented.

The scissor mechanism 14 guides the flexible shaft 100 linearly (shown in the X-direction). The guidance is realised via a connection of the shaft 100 to the scissor mechanism 14 by means of shaft receptacle 16. To this end, it is possible in particular that the shaft 100 runs through bores in the central joints 90, 91, 93, 94. On the other hand, it is in particular also possible that for example fixing devices, such as hooks and/or eyelets, which guide the shaft 100, are connected to the central joints 90, 91, 93, 94 of the scissor mechanism 14. On account of this parallel guidance in relation to the scissor mechanism 14, a guided deflection of the shaft tip 102 and the shaft 100 is realised, such that a targeted insertion of the shaft tip 102 into the entry assistance device 302 is possible. In particular, vibrations of the shaft tip in the region of the entry assistance device are hereby prevented or significantly reduced.

Instead of the shown embodiment, in which the shaft 100 is guided over the entire length by the scissor mechanism, is also possible that only the shaft tip is guided by the scissor mechanism. To this end, merely the last joint 93 of the scissor mechanism may fix the shaft 100, for example.

FIG. 4b shows the device 10 with shaft 100 from FIG. 4a in the inserted state. The endoscope 200 was guided in the direction of the entry assistance device 302, for example by means of the robot arm 402, and in this way was inserted into the shaft 100. The scissor mechanism 14, in so doing, was collapsed. This collapsing may be realised on the one hand by way of a (not shown) drive means 20 of the device 10. On the other hand, it is optionally also possible that the scissor mechanism collapses automatically, for example on account of the contact with the entry assistance device 302 and on account of the advance of the endoscope 200, or of the device 10. In particular, the assistance device 302 facilitates the insertion and/or reduces the risk of injury for the patient.

Instead of the insertion of the shaft 100 via the insertion assistance device 302, it is likewise possible in this embodiment and also in the other shown embodiments that the shaft 100 is inserted directly into a bodily orifice 502 of a patient 500.

By means of this embodiment or other embodiments of the device 10, an endoscope or an endoscope shaft 100 may be guided in particular in accordance with the embodiment of FIGS. 1, 2a and 2b. Alternatively or additionally, this embodiment or other embodiments of the device 10 may be used in particular in the embodiment from FIG. 3, such that preferably the vibration (along movement arrow 112 in FIG. 3) is prevented.

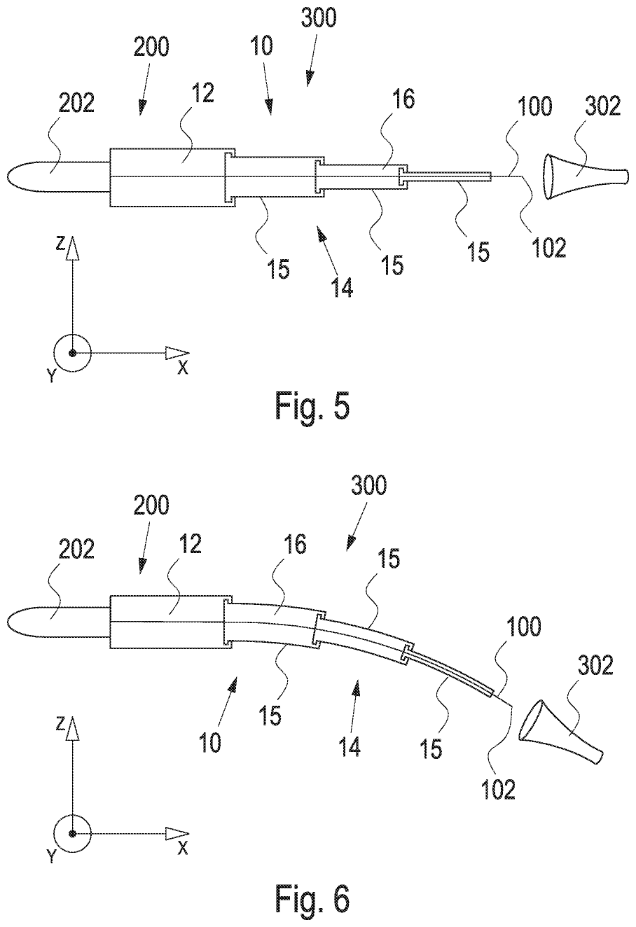

FIG. 5 shows a further embodiment of a device 10 according to the invention. Instead of the scissor mechanism 14 from FIG. 4a, the embodiment in FIG. 5 comprises a linear telescope mechanism 14. The telescope members 15 are displaceable here linearly relative to one another or inside one another. The shaft is guided by the telescope members 15, which in particular are cylindrical. These telescope members 15 in this case are in particular hollow, such that the interior of the telescope members 15 forms the shaft receptacle 16 for guidance of the shaft 100. The telescope members 15 may be moved here in particular via a drive device 20 (not shown). It is also possible that the telescope members 15 are displaceable relative to one another merely manually. In the case of this manual embodiment it is preferred that the telescope mechanism 14 retracts manually and/or automatically as the shaft 100 is removed from the patient.

FIG. 6 shows a further embodiment of the device 10 according to the invention. The embodiment from FIG. 6 corresponds here substantially to the embodiment in FIG. 5.

Instead of the linear telescope members 15 from FIG. 5, the embodiment from FIG. 6 comprises curved or arched telescope members 15. A curved or arched guidance of the shaft 100 is thus possible with the embodiment from FIG. 6. By contrast, in the embodiments from FIGS. 4a and 5, a linear deflection, in translation, is realised (shown in the X-direction).

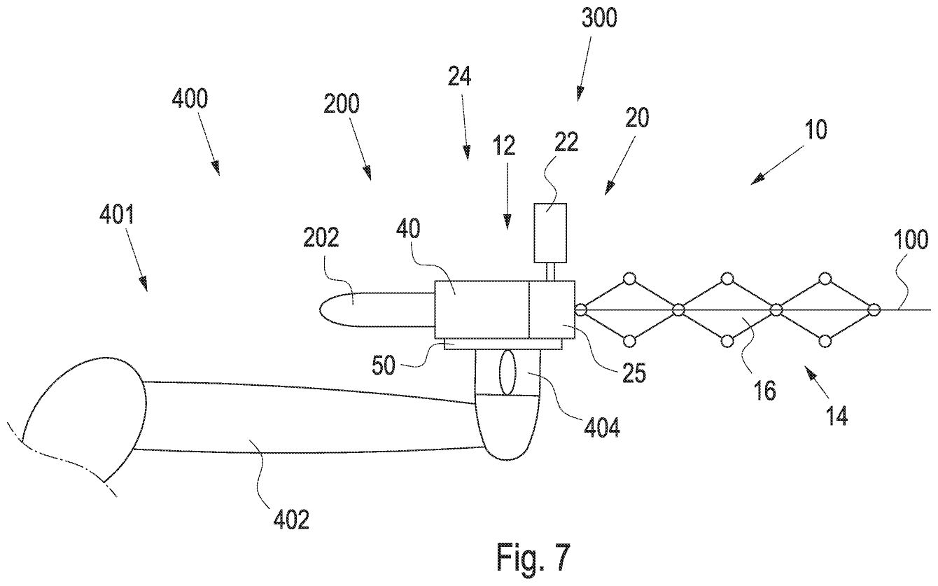

FIG. 7 shows an embodiment of an endoscope robot system 400 according to the invention with an embodiment according to the invention of an endoscopy system 300 according to the invention with an embodiment of a device 10 according to the invention.

The endoscopy system 300 comprises an endoscope 200, which is connected to the device 10 via an endoscope carrier 40 of a connection device 12. The device is in turn connected via a robot adapter 50 of the connection device 12 to a tool interface 404 of a robot arm 402 of a robot 401. The endoscopy system 300 may be deflected by the robot 401.

The shown scissor mechanism 14 guides the shaft 100 linearly (based on the embodiment from FIG. 4a). The scissor mechanism is activated here by a drive device 20, which comprises a motor 22, via a transmission device 25, such that the scissor mechanism may be extended and/or retracted. The transmission device 25 may comprise in particular a suitable transmission for the driving movement of the drive device 20. It is likewise possible that the drive device 20 (not shown) activates and therefore for example triggers an angling function of the shaft 100 by means of a coupling of actuation elements of the endoscope 200. Alternatively or additionally, it is possible (not shown) that the drive device 20 may perform a rotation of the shaft or of the entire endoscope inclusive of endoscope shaft 100. It is particularly preferred (not shown) that at least three degrees of freedom are activated by means of the device 10, in particular by means of the drive device 20, wherein these are a retraction/extension of the scissor mechanism 14, an endoscope rotation, and endoscope angling.

The robot adapter 50 may be in particular a purely mechanical fastening element. However, it particularly preferably comprises interfaces for power supply and/or communication, such that the device may be hereby controlled and/or supplied with power.

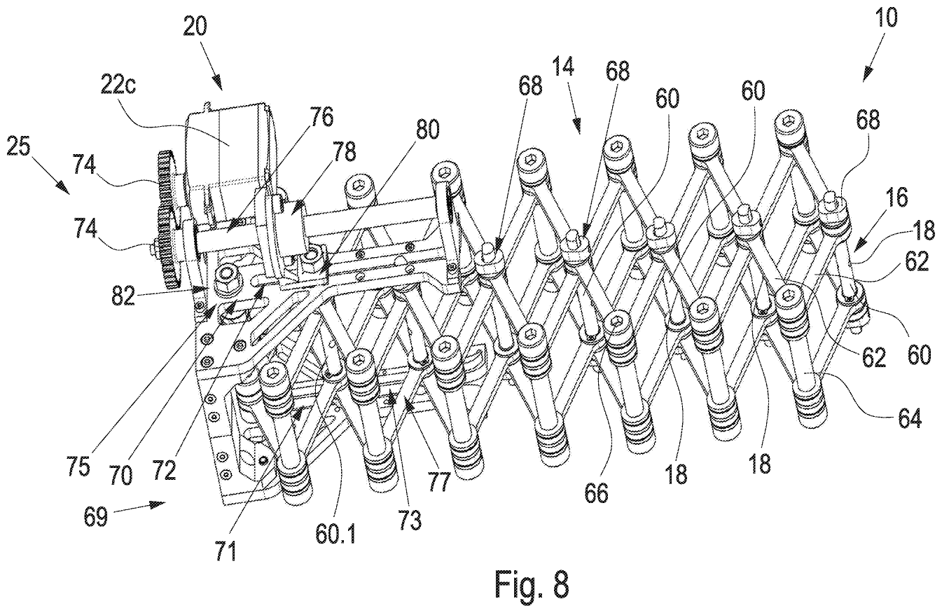

FIG. 8 shows a further embodiment of a device 10 according to the invention with three-dimensional scissor mechanism 14. The scissor bars 62 are both mounted rotatably at each of the two ends of central bars 60 and are mounted rotatably relative to one another at outer sleeves 64. Since scissor bars 62 are situated on both sides of the central bars 60, the entire scissor mechanism is stabilised in respect of the action of external forces and the force of gravity. Shoulder screws 66 screwed into the outer sleeves 64 serve as a bearing seat and hold the scissor bars 62 in position in relation to the outer sleeves 64. Alternatively (not shown), it is also possible for the outer sleeve 64 to be extended and designed such that the bearing seats are situated on the sleeves 64 and the positions are secured via retaining rings. The shown key stones 68, which are fixedly connected to the central bars 60 by means of grub screws (not shown) ensure a correct orientation of the central bars, such that all shown bores 18 in the central bars 60 are arranged in a line. This orientation is realised in particular on account of the guidance via a guide mechanism 69. This guide mechanism has two guide rails 75, 77, which are embodied by recesses 72, 72 in fastening plates 70, 71. The fastening plates 70, 71 are preferably connected releasably (not shown) to the connection device 12. On account of this possibility for release, the entire scissor mechanism 14 may preferably be separated from the connection device 12 and/or the drive device 20 for autoclaving.

The scissor mechanism 14 is activated via a transmission device 25 by a servomotor 22c of a drive device 20. The servomotor 22c to this end drives a threaded spindle 76 via two spur gear wheels 74. As a result of the rotation of the threaded spindle 76, the threaded nut 78 is displaced along the spindle, wherein the driver 80 fastened to the threaded nut entrains the first central bar 60.1. In a preferred (not shown) alternative embodiment, the first outer sleeve 64 could be guided over a circular path about the first bearing point. Since the driver 80 rests on the fastening plate 70, in particular via a sliding coating, an undesirable concomitant rotation of the threaded nut 78 with the spindle 76 is preferably ruled out. The movement of the first central bar and therefore a change to the distance of the central bars 60 from one another results in a shortening or a lengthening of the scissor mechanism 14 and therefore a deflection of the scissor mechanism. It is preferred that the shaft is guided through the bores 18 in the central bars 60, such that these bores 18 therefore represent the shaft receptacle 16.

It is preferred that at least the bores 18 in the central bars 60 are sterile, such that the endoscope shaft 100 is not contaminated. It is therefore preferred that at least the scissor mechanism 14, in particular the entire device 10, may be autoclaved. Here, it is preferred that the drive device may be removed or decoupled from the device 10, in particular without the use of tools. It is likewise preferred that the device may be separated from a robot and/or the robot adapter 50, in particular without the use of tools.

In this embodiment and other embodiments it is preferred that at least the at least one motor 22 of the drive device 20, preferably the entire drive device 20, is preferably arranged structurally independently of the scissor mechanism. It is thus possible to separate the scissor mechanism 14 from the device 10, such that this may be sterilised without the drive device 20 needing to be separated and/or formed in a manner suitable for sterilisation.

Instead of the transmission of the motor rotation by means of threaded spindle and/or spur gears, further variants of the force transmission, in particular cone gear wheels, spur gear wheels and/or wire cables, etc., may also be provided.

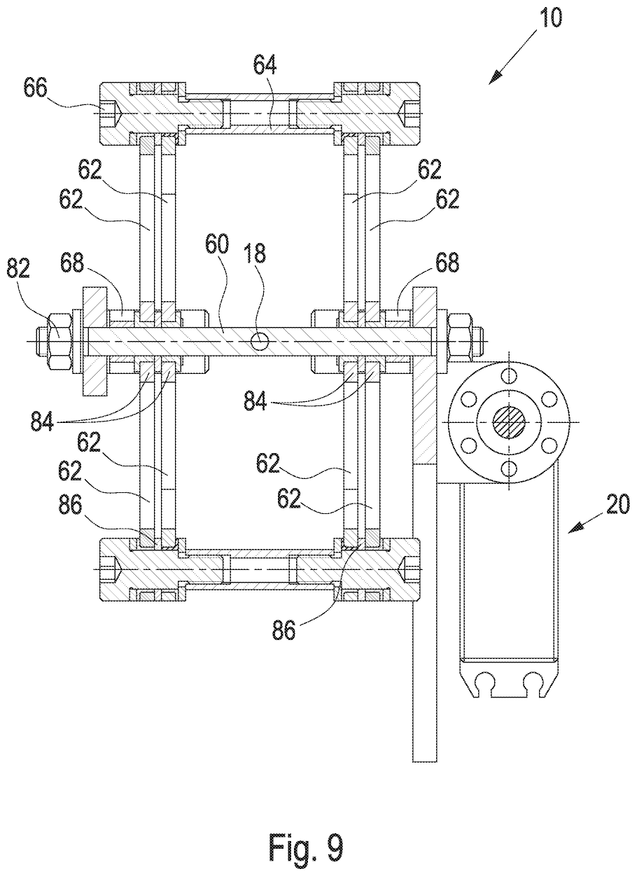

FIG. 9 shows a cross-section through the first central bore 60.1 in accordance with the embodiment in FIG. 8.

The endoscope shaft 100 is preferably guided in the bore 18 in the centre of the central bar 60.

The scissor bars 62 are both mounted rotatably on both ends of the central bars 60 via sliding bearings 84 and thrust washers 86 and are mounted rotatably relative to one another at the matching faces of the shoulder screws 66 screwed into the outer sleeve 64. Alternatively (not shown), it is also possible for the outer sleeve 64 to be lengthened and thus designed such that the bearing tips are situated on the sleeves 64 and the positions are secured via retaining rings.

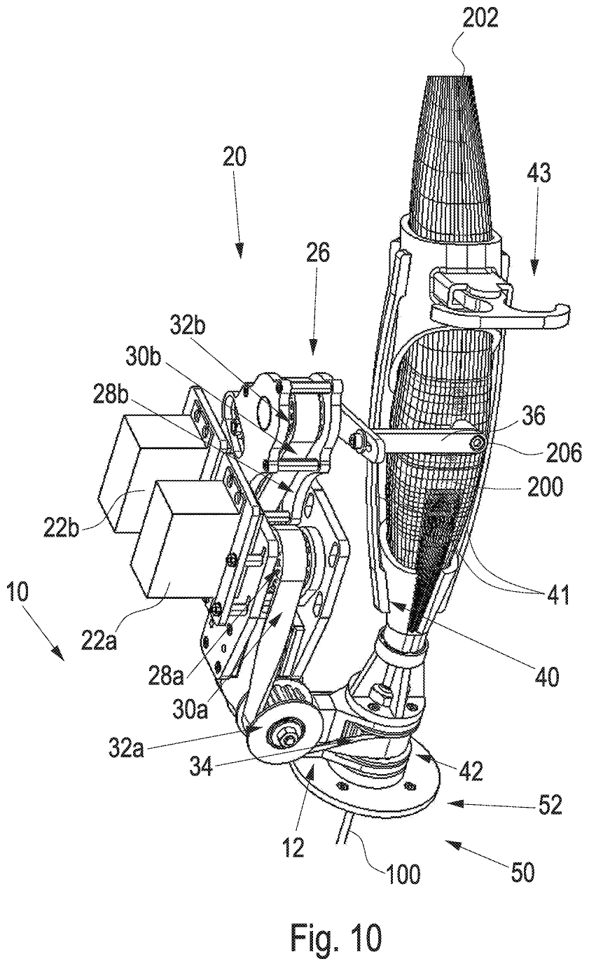

FIG. 10 shows a further embodiment of a device 10 according to the invention without the presented bridging device 14.

The endoscope 200 is connected to the device 10 via an endoscope carrier 40 of a connection device 12. As is shown, the endoscope carrier comprises, in particular flexible, carrier bars 41, which for example are pressed against the endoscope gripper unit 202 by means of a detent closure 43.

The device additionally has a drive device 20, in particular for rotation of the entire endoscope 200 inclusive of endoscope shaft 100 about the longitudinal axis of the endoscope shaft 100 and for angling of an endoscope tip 102 (not shown).

In particular for rotation of the entire endoscope 200 inclusive of endoscope shaft 100, the drive device 20 comprises a first servomotor 22a, which drives a toothed belt 30a via a gear wheel 28a. The toothed belt 30a is tensioned via a deflection pulley 32a and is deflected towards the stationary gear wheel 34. Since a drive carrier 42 is mounted rotatably in relation to the gear wheel 34 (see in particular FIG. 11), the entire device 10 rotates about the longitudinal axis of the endoscope shaft 100. In particular, it is possible here that a bridging device 14 (not shown) is concomitantly rotated, or that the bridging device 14 is not rotated, wherein this is implemented in particular with appropriate mounting of the bridging device 14.

In order to angle the endoscope tip 102, the drive device 20 preferably comprises a second servomotor 22b, the movement of which is transmitted to a lever 36 via a drive-side gear wheel 28b, the toothed belt 30b, and an output-side gear wheel 32b. The lever 36 transmits the movement to the actuation element 206 of the endoscope 200. It is hereby preferred in particular that the axis of rotation of the lever 36 coincides as exactly as possible with the axis of rotation of the actuation element 206 of the endoscope 200.

The device is connected to a robot 401 preferably via a robot adapter 50, in particular via a suitable interface 52.

Instead of the shown connection of the endoscope 200 to the device 10, it is in particular also possible to provide a connection via screwed U-bolts and/or union nuts. It is particularly preferred that the endoscope is connected to the device 10 via fastening variants of the endoscope carrier 40 which do not require any additional tools for the fastening of the endoscope 200. In addition, it is preferred that the endoscope carrier 40 is suitable for sterilisation, in particular suitable for autoclaving. Alternatively to the shown embodiment of the servomotor 22a, it is possible to mount the servomotor 22a in a stationary manner at the interface 52. As an alternative to the toothed belts 30a, 30b, it is in particular possible to use other variants for force transmission, in particular cone gear wheels, spur gear wheels and/or wire cables, etc.

In an alternative embodiment it is likewise possible to mount the second servomotor 22b in a stationary manner at the interface 52 and to embody the drive for transmission of the lever movement in particular via wobble plates and/or driven, rotatable sleeves with a slot for the lever of the actuation element 206.

It is also alternatively possible to provide just one motor 22 having corresponding couplings and/or transmissions.

FIG. 11 shows a cross-section through the embodiment from FIG. 10.

The stationary gear wheel 34 is fixedly connected to a hollow axis 406 via a grub screw 38, wherein the hollow axis 406 is in turn fixedly connected to the interface 52. Drive carriers 42, 42′, 42″ and the endoscope carrier 40 connected thereto are mounted movably in relation to the interface 52 via ball bearings 44′, 44′. Instead of ball bearings, other bearing forms are possible, for example sliding bearings. An endoscope shaft 100 (not shown) is guided through the central opening 39, which is formed by central recesses in the endoscope carrier 40, the drive carrier 42, and the interface 52.

It is preferred that the opening 39 is sterile in order to avoid contamination of the endoscope shaft 100. To this end, it is preferably possible to insert a sterile disposable sleeve (not shown) into the opening 39.

Instead of the shown grub screw 38 in order to prevent rotation of the gear wheel 34, other variants are also possible, such as feather keys and/or a polygonal profile at the drive carrier and also at the gear wheel.

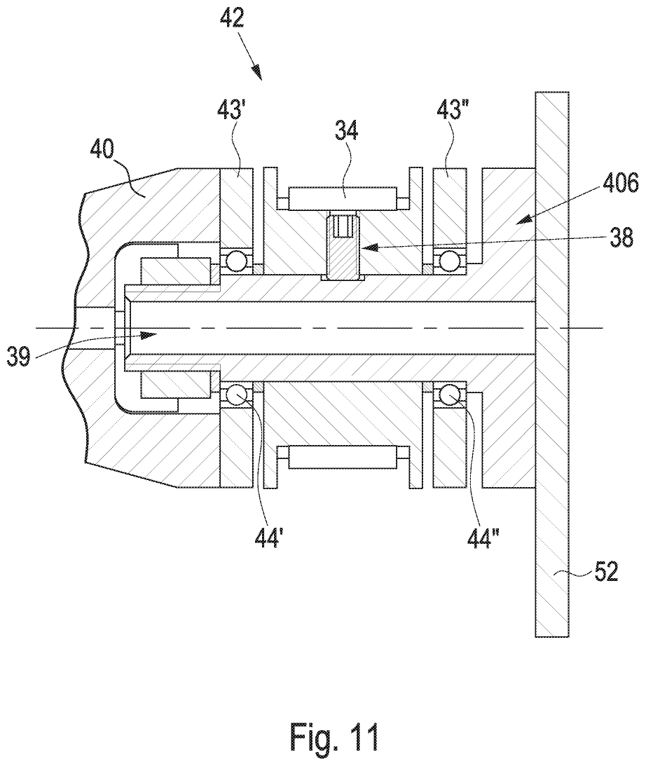

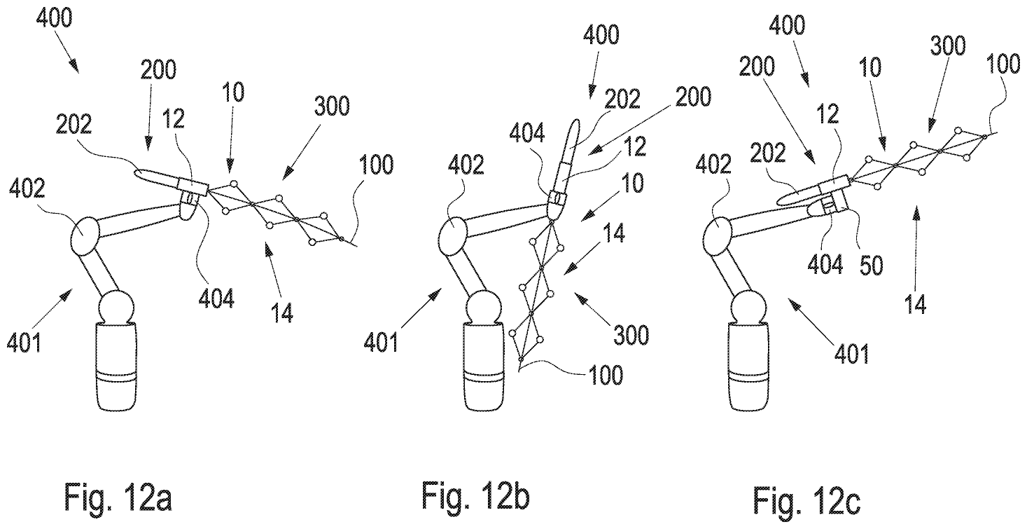

Various embodiments of the endoscopy robot system 400 according to the invention with endoscopy systems 300 according to the invention with device 10 according to the invention are shown in FIGS. 12a, 12b and 12c. The endoscopy system 300 corresponds here substantially to the embodiment from FIG. 4a.

FIGS. 12a, 12b and 12c show various connection types of the endoscopy system 300 or of the device 100 on a robot 401. In particular, the optimal connection type is dependent on the robot kinematics and the desired intraoperative movability of the endoscope.

FIG. 12a shows a right-angled connection of the device 10 via connection device 12 to the tool interface 404 of the robot 401.

FIG. 12b shows a coaxial connection of the device 10 to the tool interface 404 of the robot 401. The device 10 is preferably connected here to the tool interface 404 via the connection device 12, wherein the endoscope shaft runs through a hollow axis in the tool interface 404. To this end it is preferred that the hollow axis of the tool interface 404 has a sufficiently large diameter to receive and/or allow passage of the device 10.

FIG. 12c shows a parallel connection of the device 10 to the tool interface 404, wherein the connection is in particular true-parallel. The connection is established here by way of the connection device 12, which comprises a robot adapter 50, which is connected at the end face to the tool interface 404, wherein the rest of the device 10 is connected at right angles to the robot adapter 50.

1. A device for guiding a medical flexible shaft to a body into which a shaft is to be inserted, comprising:
a bridging device that is length-variable, and
a shaft receptacle connected to the bridging device for fixing the shaft to the bridging device,
wherein the bridging device prevents vibration of the shaft at least at a bodily insertion orifice.

2. The device according to claim 1, wherein the bridging device comprises:
a scissor mechanism and/or
a telescope mechanism and/or
a bellows mechanism.

3. The device according to claim 1, wherein the shaft receptacle is formed in such a way that the shaft may be received at least at a distal end of the bridging device,
wherein the shaft receptacle is formed in such a way that the shaft may be received over an entire length of the bridging device, such that the shaft is guided parallel to the bridging device.

4. The device according to claim 1, wherein the shaft receptacle is designed in such a way that the shaft runs at least in part within the bridging device.

5. The device according to claim 1, further comprising: a drive device comprising at least one motor, the drive device configured to activate the device and/or the shaft and/or an endoscope, wherein the drive device may be separated manually from the device.

6. The device according to claim 5, wherein the drive device is configured to activate the bridging device.

7. The device according to claim 5, wherein the drive device activates an endoscope comprising the shaft, and wherein the drive device rotates the shaft indirectly or directly and/or activates at least one shaft function of the endoscope.

8. The device according to claim 1, further comprising a connection device for connecting the device to the shaft and/or a shaft base connected to the shaft.

9. The device according to claim 8, wherein the connection device comprises:
a coupling for activating a shaft function and/or an endoscope function based on activating actuation elements of the endoscope, and/or
a shaft and/or an endoscope carrier for structural connection of the shaft or of the endoscope to the device.

10. The device according to claim 8, wherein the connection device comprises:
a robot adapter for connecting the device to a robot, wherein the robot adapter comprises at least one interface for electrical and/or hydraulic energy transmission and/or communication.

11. The device according to claim 1, wherein
the bridging device extends from a proximal end of the shaft to a receiving bodily orifice.

12. The device according to claim 1, further comprising: a control device comprising at least one wireless and/or wired transmitting and/or receiving device for remote control of a drive device.

13. The device according to claim 1, wherein the device comprises materials suitable for autoclaving.

14. A medical endoscopy system, comprising,
a device according to claim 1, and
a flexible endoscope,
wherein the bridging device receives an endoscope shaft and guides the endoscope shaft from a proximal shaft end to a receiving bodily orifice.

15. The medical endoscopy system according to claim 14, further comprising a support for fixing the endoscopy system in a room, wherein the support is a movable support.

16. The medical endoscopy system according to claim 14, further comprising:
an operating device comprising at least one input device and/or at least one imaging device for remote control of the device, and/or
an entry assistance device at the receiving body.

17. A medical endoscopy robot system, comprising
an endoscopy system according to claim 14, and
a robot arm,
wherein the device for guiding a medical flexible shaft is connected to a tool interface of the robot arm.

18. A method for guiding a medical flexible shaft to a receiving body, comprising:
guiding a shaft tip forwards and/or backwards via a bridging device to the receiving body.

19. The method according to claim 18, wherein a flexible shaft is guided from a shaft base to the receiving body along a linear line.

20. The method according to claim 18, wherein the method is performed by a device according to claim 1.
